(19) United States
(12) Patent Application Publication
(10) Pub. No.: US 2002/0156424 A1
(43) Pub. Date: Oct. 24, 2002

(54) Needle covering member and syringe equipped therewith

(76) Inventors: Hitoshi Suzuki, Yokohama-shi (JP); Seiichi Takigawa, Yokohama-shi (JP)

Correspondence Address: KUBOVCIK & KUBOVCIK, SUITE 710, 900 17TH STREET NW, WASHINGTON, DC, 20006

(21) Appl. No.: 10/126,615

(22) Filed: Apr. 22, 2002

(30) Foreign Application Priority Data
Apr. 24, 2001 (JP) 2001-125234
Jul. 31, 2001 (JP) 2001-230971

(51) Int. Cl. A61M 5/32

(52) U.S. Cl. 604/192

(57) ABSTRACT

In order to provide a needle covering member which can protect a needle tip, when carried to a patient with its syringe containing fluid to be injected, and which can retard re-exposure of the needle having been covered with the needle covering member even under external loads, the protector (32) of the needle covering member includes a housing opening (32a), a stopper portion (34a) for stopping the protector (32) from turning in such a direction as to expose the needle (10) covered with the protector, once the protector (32) has been turned and covered the needle (10), even under external loads, and a pair of manipulation pieces (34) for releasing the protector (32) from the stopped state, to which it has been brought by the above stopper portion (34a), so as to make the needle (10) having been covered with the protector (32) available, the above stopper portion (34a) being formed at the housing opening (32a) side ends of the pair of manipulation pieces (34) and intersecting the needle (10) or a needle hub (11) in the interior of the protector (32).

### BACKGROUND OF THE INVENTION

1. Field of the Invention

The present invention relates to a needle covering member for covering the tips of used needles including indwelling needles and a syringe equipped therewith.

2. Description of the Related Art

Used needles carry the risk of exposing people to, for example, AIDS or hepatitis infection. In order to avoid this risk, there have been provided various types of injectors equipped with protectors for protecting people from accidental contact with the tip of a used needle.

One type of techniques for reducing such a risk is to provide an injector with a protector connected to a needle hub via a hinge. This type of technique is disclosed in, for example, JP 4-244167 A (Safety Device Used for Needles), JP 2989937 B2 (Safety Device Used with Needles), JP 2882470 B2 (Protective Barrier Assemblage for Needle Element) and JP 2921562 B2 (Needle Shielding Assemblage).

Analyzing these prior arts, they can be divided into two subtypes.

The first subtype is the one in which a used needle is fixed in a protector (housing 14) so as not to re-expose after being housed in the protector, as disclosed in JP 4-244167 A (Safety Device Used for Needles) and JP 2989937 B2 (Safety Device Used with Needles). The device disclosed in JP 4-244167 A is shown in FIG. 14. In these prior arts, though a safety device 30 is incorporated in a needle 10 before packed, in order to prevent the needle 10 from exposing at the time of unpacking, a cap as a separate member (not shown in the figure) is required.

On the other hand, the second subtype is the one in which the needle tip of the injector containing the fluid to be injected can be protected while being carried to a patient, as disclosed in JP 2882470 B2 (Protective Barrier Assemblage for Needle Element) and JP 2921562 B2 (Needle Shielding Assemblage). In this subtype, a protective member can be opened and closed, as shown in FIG. 15. This subtype of an assemblage 30 is also incorporated in a needle 10 before packed. In this subtype, a cap can be omitted because it is possible to prevent the needle tip from exposing at the time of unpacking when having been packed so as not to expose.

By the way, In the subtype in which a needle is fixed in a protector, the needle tip cannot be sufficiently protected when the syringe containing the fluid to be injected is carried to a patient. Further, since a cap as a separate member is required, the large number of the members should be discarded.

On the other hand, in the subtype in which protective member can be opened and closed, though the needle tip can be protected when the syringe containing the fluid to be injected is carried to a patient, there still remains a few problems about engaging members (engaging pins and holes) for covering a used needle.

Specifically, since the plane on which a protector turns and the direction in which loads are applied when disengaging the engagement are on the same plane, the protector may slip off from the needle when external loads are applied to the protector in such a direction as to disengage the engagement. When the injector protected by the protector is held in a hand, something that is collided against the protector may give the protector external loads in such a direction as to allow the protector to slip off from the syringe. In order to avoid such a situation, a protector and engaging members must be formed firmly and thereby an assemblage itself becomes larger and heavier. This results in increase of the number of members to be discarded.

### SUMMARY OF THE INVENTION

The problem to be solved by the present invention is how to provide a technique in which the needle tip, when the syringe containing the fluid to be injected is carried to a patient, can be protected and, furthermore, in which the covered needle is hard to re-expose even under external loads.

Accordingly, the object of the present invention is to provide a needle covering member which can protect the needle tip, when the syringe containing the fluid to be injected is carried to a patient, and, furthermore, by which the covered needle is hard to re-expose even under external loads, and also to provide a syringe equipped with the needle covering member.

The present invention has been made to accomplish the above object.

The present invention relates to a needle covering member (30) comprising a fixing portion (31) for fixing the needle covering member in the vicinity of the tip of a syringe (20), a protector (32) for housing and covering a needle (10), and a connecting portion (31a) for connecting the protector (32 and the fixing portion (31) to each other.

The protector (32) comprises a housing opening (32a) through which the needle (10) gets in and out, a stopper portion (e.g. a needle-supporting portion 34a of a pair of manipulation pieces 34) for preventing the protector (32) from turning in such a direction as to expose a needle (10) even under external loads when the protector is turned toward the housing opening (32a) and covers the needle (10), and a manipulation piece (34) for releasing the protector (32) from the stopper portion (34a) so that the needle (10) covered with the protector (32) may be exposed through the housing opening (32a) to be used.

The stopper portion (34a) is formed at an end of the manipulation piece (34) near to the housing opening (32a) and intersects the needle (10) or a needle hub (11) in the interior of the protector (32).

The phrase “an end of the manipulation piece (34) near to the housing opening (32a)” herein used does not strictly mean the end of the manipulation piece, but includes the case in which the stopper portion is positioned on the manipulation piece when the needle (10) is surely housed in the protector (32) without exposing and is surely released with no trouble.

Moreover, the phrase “the vicinity of the tip of a syringe” includes not only the tip portion (22) of the syringe, but also the vicinity of the tip of a barrel portion (23) and the needle hub (11).

The stopper portion may intersect both the needle (10) and the needle hub (11).

In the present invention, the needle covering member (30) is fixed to an injector in the vicinity of the tip portion of the syringe (20) using the fixing portion (31).

After the fluid is drawn through the needle (10), the needle (10) is housed in the protector (32) through the housing opening (32a) using the connecting portion (33) which is connected to the fixing portion (31). Once the needle (10) is housed in the protector (32), the protector (32) is not allowed to turn in such a direction as to expose the needle (10) even under external loads by making the stopper portion (34a) intersect the needle (10) or the needle hub (11).

When using the needle (10) housed in the protector (32) again, the stopped state by the stopper portion (34a) is released the protector (32) from, to which it has been brought, by manipulating the manipulation piece (34) and exposing the needle (10) covered with the protector (32) through the housing opening (32a). For example, after the needle (10) with the syringe containing the fluid is protected with the protector (32) and carried to a patient, the stopped state by the stopper portion (34a) is released. The protector (32) can protect the needle (10) with the syringe containing the fluid while the needle (10) is carried to a patient and then exposing the needle again for use. Thus, injection into a patient can be done.

After injection, in order to prevent a needle prick accident, the needle (10) is housed again in the protector (32), and the stopper portion (34a) is made to intersect the needle (10) so that the needle (10) may not be exposed.

If the needle covering member (30) is fixed to a needle when packing the needle, the exposure of the needle at the time of unpacking immediately before used can be prevented. Accordingly, a cap as a separate member is not necessary. Thus, the total amount of the wastes can be reduced compared with the prior arts which require a cap.

In the needle covering member of the present invention, the protector (32) deforms elastically so that the needle (10) may easily get out of the housing opening (32a) when manipulating the manipulation piece (34) to release the stopped state by the stopper portion (34a).

The state in which “the protector (32) deforms elastically so that the needle (10) may easily get out of the housing opening (32a) when manipulating the manipulation piece (34)” is accomplished, for example, by means in which the whole protector (32) is formed of a thin thermoplastic resin and has a structure so as to widen the housing opening (32a) by manipulating the manipulation piece (34) or by means in which an auxiliary manipulation piece which interlocks with manipulation of the manipulation pieces (34) and pushes the needle (10) toward the housing opening (32a) is provided.

Forming the whole protector (32) of a thin thermoplastic resin, the total amount of wastes related to the needle covering member can be reduced.

Thus, the needle covering member (30) can release the stopped state by the stopper portion (34a) by manipulating the manipulation piece (34). Then the protector (32) deforms elastically so that the needle (10) may easily get out of the housing opening (32a).

The needle covering member of the present invention may include a needle-stabilizing-functioning portion (e.g. a needle contact portion 35) for housing the needle (10) in the interior of the protector (32) in a position where the needle is easily released.

In this needle covering member, the needle (10) is housed in the interior of the protector (32) in a position where the needle is easily released, due to the presence of the needle-stabilizing-functioning portion (35).

Further, in the needle covering member (30) of the present invention, the protector may include a release-retarding-functioning portion (e.g. real-stopping projections (32f), engaging portion (36e), or a connecting plate (34c)) for retarding the release of the stopped state by the manipulation piece.

In this needle covering member, after injection into a patient and covering the needle with the protector (32), the release-retarding-functioning portion retards the movement of manipulation piece (34). Thereby the protector (32) does not turn in such a direction as to expose the needle (10).

In the needle covering member of the present invention, the stopper portion may intersect the needle around the middle of its length in the interior of the protector (32).

The phrase “around the middle of its length” herein used concerning “the stopper portion (34a)” means the portion of the needle (10) excluding its tip portion and the needle hub (11). In a more preferable form of the present invention, the phrase “around the middle of its length” excludes the root of the needle (10) or its vicinity. The reason is that the loads applied to the stopper portion can be much more reduced even if external loads are applied to the protector to slip off.

In this needle covering member, when external loads are applied to the protector in such a direction as to bring the needle from the covered state to the uncovered state, the loads applied to the stopper portion can be much more reduced compared with other needle covering members in which the stopper portion is provided at the root of the protector or its vicinity, because the stopper portion is provided not at the root of the protector or vicinity, but around the middle of the protector. Thus, the needle tip can be more effectively prevented from being exposed.

Further, in the interior of the protector (32), the stopper portion (34a) comes in contact with the middle portion of the needle (10) or its vicinity and not with the needle tip. Therefore, it is preferable from the hygienic point of view.

In the needle covering member of the present invention, the manipulation flow line, which indicates the direction in which loads are applied to the manipulation piece so as to release the stopped state by the stopper portion, may not be on a plane on which the protector turns.

In this needle covering member, the manipulation flow line, which indicates the direction in which loads are applied to the manipulation piece so as to release the stopped state by the stopper portion, is not on a plane on which the protector turns. Therefore, the accidental exposure of the needle point can be prevented more effectively.

The other invention relates to a syringe equipped with the above-mentioned needle covering member including a piston which enables the discharge and suction of fluid, a barrel portion for holding the fluid, and a tip portion provided at the tip of the barrel portion.

### BRIEF DESCRIPTION OF THE DRAWINGS

FIG. 1 shows a perspective view, a sectional view of the main part and a side view of the first embodiment.

FIG. 2 shows another perspective view and another side view of the first embodiment.

FIG. 3 shows variations in shape of a protector and a manipulation piece.

FIG. 4 shows variations around the manipulation piece.

FIG. 5 shows variations in shape of manipulation piece and a variation in which a plurality of manipulation positions are provided.

FIG. 6 shows perspective views of another embodiment in which highly functional requirements are realized.

FIG. 7 shows a variation around the manipulation piece.

FIG. 8 shows another variation around the manipulation piece.

FIG. 9 shows still another variation around the manipulation piece.

FIG. 10 shows partial enlarged views sectional views of the embodiment shown in FIG. 9.

FIG. 11 shows a view showing the manipulation piece, being manipulated, of the embodiment shown in FIG. 9.

FIG. 12 shows the protector, being pushed on its sides, of the embodiment shown in FIG. 9.

FIG. 13 shows another variation around a manipulation piece.

FIG. 14 shows a view of the safety device disclosed in JP 4-244167 A.

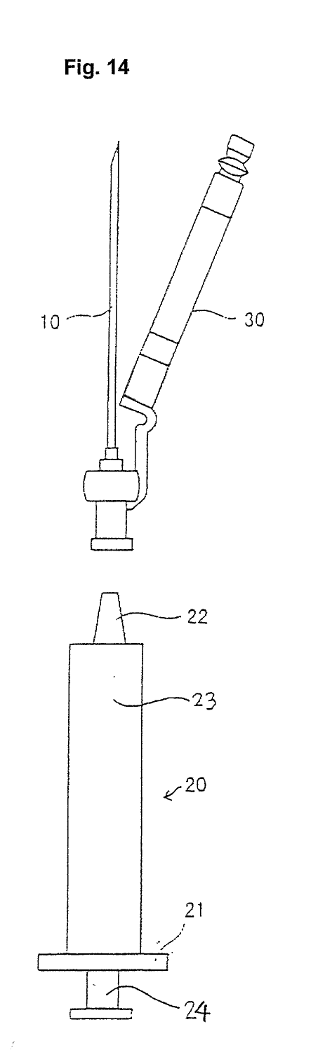

FIG. 15 shows a view of the needle shielding assemblage disclosed in JP 2921562 B2.

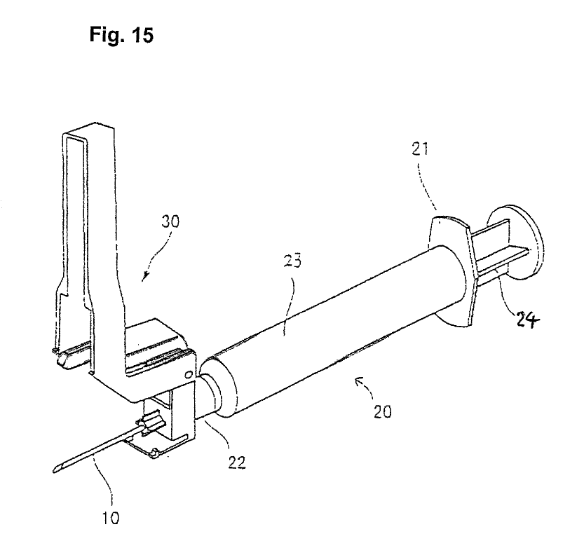

### DETAILED DESCRIPTION OF THE PREFERRED EMBODIMENTS

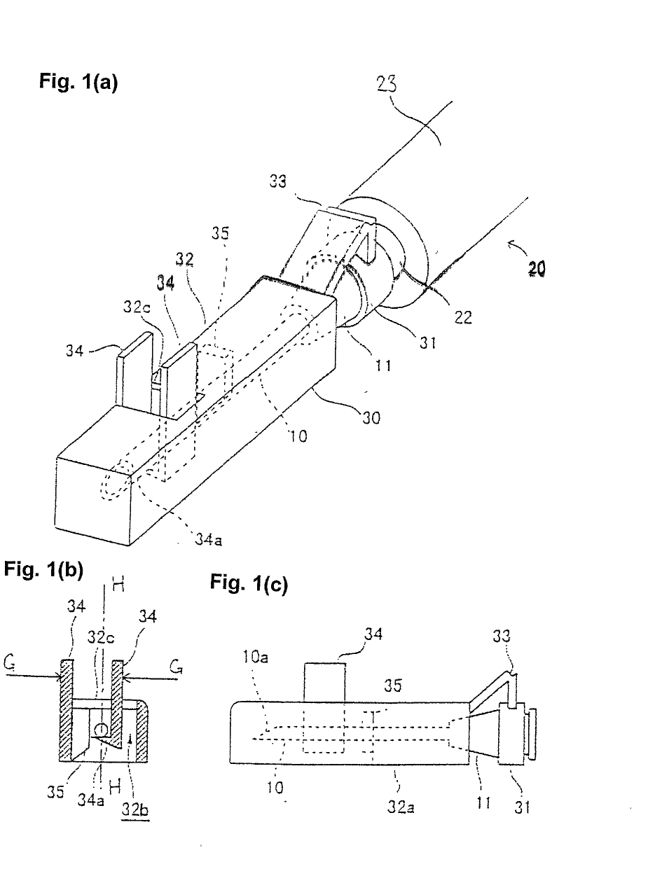

In the following, the present invention will be described in further detail in terms of its preferred embodiments with reference to the accompanying drawings. The drawings herein used will be FIGS. 1 to 13.

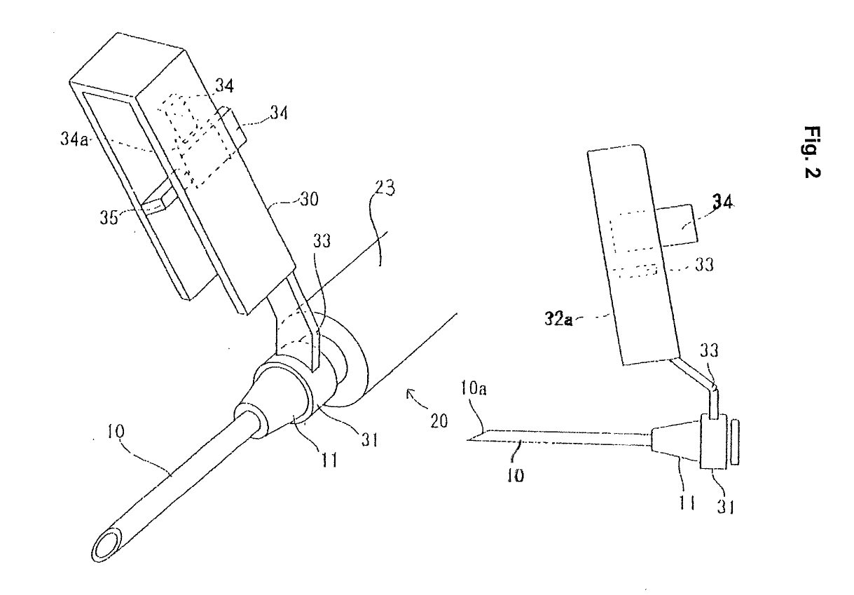

FIG. 1 shows a perspective view, a sectional view of the main part and a side view of a needle covering member of the first embodiment. FIG. 2 shows a perspective view and a side view which illustrate a state where a protector 32 in the first embodiment shown in FIG. 1 does not house a needle 10.

A needle covering member 30 of this embodiment is fixed to a tip portion 22 of a syringe 20. The needle covering member includes a fixing ring 31 for fixing the needle covering member to a needle hub 11 of a needle 10, a protector 32 for housing and covering the needle 10, and a connecting portion 33 for connecting the protector 32 and the fixing ring 31 to each other.

The above protector 32 is a box-shaped protector with its bottom opened, the opening formed in the bottom is a housing opening 32a for getting the needle 10 in and out of the protector 32 and the inside space of the housing opening 32a is a housing space 32b. Around the middle of the length on the surface opposite to the housing opening 32a is provided a manipulation opening 32c which is in communication with the housing space 32b. For the manipulation opening 32c a pair of manipulation pieces 34 are provided. One of the manipulation pieces 34 has a hooky end which faces the needle 10 and the portion opposite to the hooky end projects from the housing space 32b. In the position opposite to the above manipulation piece 34 is provided the other manipulation piece 34 which is formed in such a manner as to extend part of the side surface of the protector 32 upward. Both manipulation pieces are integrated into the protector 32 at the boundary between themselves and the manipulation opening 32c.

The hooky part described above functions as a stopper portion 34a capable of hooking the middle of the needle 10 or its vicinity onto it when the protector 32 has been turned relative to the needle 10 toward the side near to the housing opening 32a and covered the needle 10. The protector 32 is designed to allow the spacing between the hooky part and a needle contacting portion 35 to become smaller than the diameter of the needle 10 when the protector 32 is turned relative to the needle 10 toward the side near to the housing opening 32a.

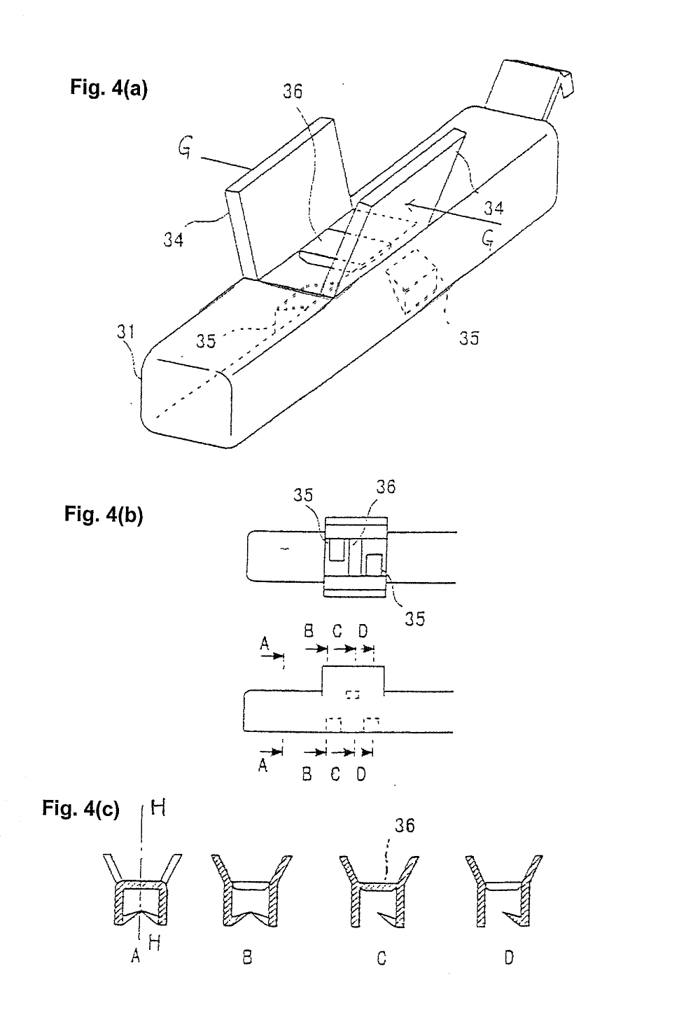

It is also possible to position two facing hooky parts in such a manner as to be staggered along the axis of the needle 10, as shown in FIG. 4. Also in this case, the protector 32 is designed to allow the two hooky parts to hook the middle of the length of the needle 10 or its vicinity onto them.

In state where the needle-supporting portion 34a, as a stopper portion as described above, hooks the middle of the needle 10 or its vicinity, the protector 32 does not turn in such a direction as to expose the needle 10 even under external loads, unless the protector itself is deformed or the needle-supporting portion 34a or the needle 10 breaks.

Since the needle-supporting portion 34a supports the needle 10 at a position away from a fixing portion 31 around which the protector 32 turns, the loads applied to the stopper portion can be reduced, particularly when external loads are applied to the tip portion of the protector 32 or its vicinity, compared with the structures in which the protector 32 is stopped near the fixing portion 31, which is in the vicinity of the root of the protector (e.g. the needle shielding assemblage disclosed in JP 2921562 B2 which has been taken as example of the prior arts). As a result, the needle tip can be more effectively prevented from being exposed.

If a needle covering member is formed to have a structure in which a protector is stopped near a fixing portion 31, and at the same time, to withstand heavy external loads, the total volume and mass of the needle covering member will inevitably become large. This results in increase in its raw materials and wastes. The needle covering member of this embodiment contributes to reducing the consumption of raw materials because it adopts the above described structure.

Further, the needle covering member of this embodiment includes a needle-stabilizing-functioning portion for stabilizing the needle (10) in such a position as to make it easier to release the protector (32) from the stopped state. In particular, the needle covering member of this embodiment includes a needle contacting portion 35 which is positioned near to the fixing portion 31 relative to the manipulation pieces 34 and projects toward the housing space 32b of the protector 32. Although the housing space 32b is large for the thickness of the needle (10), the presence of the needle contacting portion 35 enables positioning of the needle (10) housed in the housing space 32b without offsetting.

In the pair of manipulation pieces 34, if the parts projecting from the manipulation opening 32c are moved in such a manner as to approach each other, the needle-supporting portion 34a at the opposite end moves away from the needle (10) and thereby the protector 32 can be released from the stopped state. The needle (10) in the inside space 32b of the protector 32 is housed in such a position as to make it easier to release the protector (32) from the stopped state, due to the presence of the needle-stabilizing-functioning portion 35, and thereby it is easy to expose from the inside space 32b.

If loads are applied to parts of the pair of manipulation pieces 34 which project from the manipulation opening 32c from the outside in, as shown in FIG. 1(B), the protector 32 can be released from the stopped state as described above. On the other hand, however, since the manipulation flow lines G, which indicate the direction in which loads are applied, are not on a plane H on which the protector turns, the accidental exposure of the needle can be prevented effectively. In this embodiment, the manipulation flow lines G are perpendicular to the plane H on which the protector turns.

The first embodiment described above can be provided as an assemblage of an unused needle 10, a syringe and a needle covering member, and the assemblage can be packed in such a state that the protector 32 of the needle covering member covers the needle 10 and the needle-supporting portion 34a prevents the needle from being exposed. This makes it unnecessary to protect the needle 10 with a separate member such as cap, leading to reduction of raw materials and wastes accompanying cap manufacture.

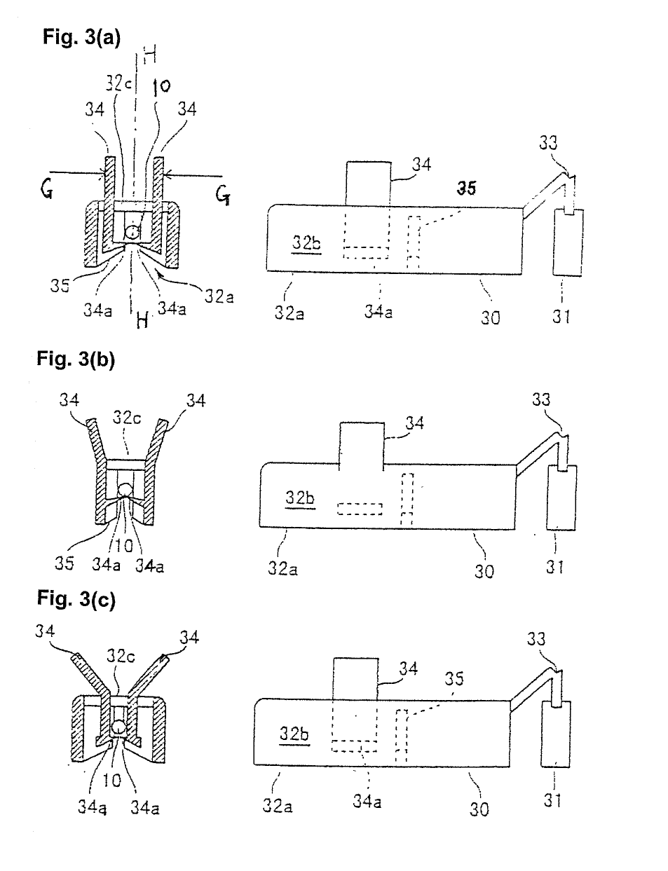

FIG. 3 shows views of variations in shape of protector 32 and manipulation pieces 34.

The manipulation pieces 34 shown in FIG. 3(A) consist of flat plates. In this case, the protector 32 becomes wide and the size of each stopper portion 34a becomes large in the horizontal direction.

In the manipulation pieces 34 shown in FIG. 3(B), the knobs serve as the side plates of the protector 32 as well and the spacing between the two knobs is slightly broadened toward their upper ends. In this case, the protector 32 is allowed to be narrow. In this type of needle covering member, since the spacing between the pair of manipulation pieces 34 of the protector 32 is slightly broadened toward their upper ends, even if the needle-supporting portions 34a are not at the strict ends of the respective manipulation pieces 34 but on the manipulation pieces 34, as shown in FIG. 3(B), the protector 32 can be adequately released from the stopped state at the time of manipulation.

In the manipulation pieces 34 shown in FIG. 3(C), the spacing between the two knobs is largely broadened toward their upper ends. In this case, the protector 32 becomes wide and the size of each stopper portion 34a becomes small in the horizontal direction.

Also in this embodiment, since the manipulation flow lines G, which indicate the direction in which loads are applied to the manipulation pieces, are not on a plane H on which the protector turns, the accidental exposure of the needle can be prevented effectively. Also in this embodiment, the manipulation flow lines G are perpendicular to the plane H on which the protector turns.

FIG. 4 shows variations of pair of manipulation pieces and vicinity when adopting a thin thermoplastic resin which easily undergoes elastic deformation as a material for the protector 32. In this embodiment, the hooky parts facing each other are positioned in such a manner as to be staggered along the axis of the needle 10 and an auxiliary manipulation piece 36 straddling the pair of manipulation pieces, which face each other, is provided halfway between the two staggered hooky parts. The presence of this auxiliary manipulation piece 36 allows the needle-supporting portion 34a to broaden when manipulating the pair of manipulation pieces 34, and at the same time, allows the housing opening of the protector 32 to largely broaden, whereby the needle housed in the protector 32 becomes easy to expose, leading to improvement in manipulability.

Also in this embodiment, since the manipulation flow lines G, which indicate the direction in which loads are applied to the manipulation pieces, are not on a plane H on which the protector turns, the accidental exposure of the needle can be prevented effectively. The manipulation flow lines G are perpendicular to the plane H on which the protector turns.

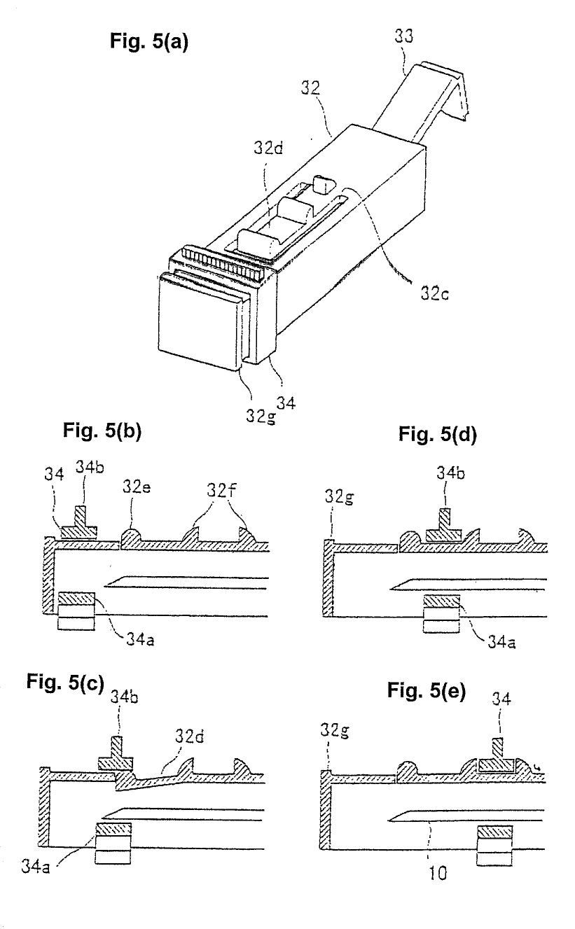

FIG. 5 shows a variation in shape of manipulation piece 34 and that of protector which provide a plurality of manipulation positions. The manipulation piece 34 is a four-sided ring circling the protector 32 and includes a manipulation knob 34b on its top surface and a needle-supporting portion 34a, as a stopper portion, on its housing opening side surface which goes into the housing space of the protector 32 and approaches a needle 10.

The protector 32 includes a manipulation opening 32c formed by cutting away a rectangular with its one side lost in its top surface, a tongue piece 32d surrounded with the manipulation opening 32c, and an end stopper 32g for preventing the manipulation piece 34 from dropping from the opposite end to the end near to the connecting portion 33. The tongue piece 32d includes a temporary-stopping projection 32e whose cross-section is semicircular; and real-stopping projections 32f which face each other away from the temporary-stopping projection 32e and serve as a release-retarding-functioning portion, in this order from the side near to the end stopper 32g.

The manipulation piece 34 and the protector 32 having been formed into such shapes provide 3 types of manipulation positions. FIGS. 5(A) and (B) show the state in which the manipulation piece 34 is positioned between the end stopper 32g and the temporary-stopping projection 32e to ensure the action of housing a needle 10 or of exposing it again. When manipulation piece 34 is in this position, the needle tip 10 has not reached the needle-supporting portion 34a yet.

FIGS. 5(C) and (D) show the states in which the manipulation piece 34 is moving to the temporarily stopping position and has moved to it, respectively. This temporarily stopping position is a position in which the needle tip 10 is protected on the assumption that it is exposed again. Once the upper portion of the manipulation piece 34 climbs over the temporary-stopping projection 32e, the part of the needle 10 slightly away from the point toward the root reaches the needle-supporting portion 34a. Since the temporary-stopping projection 32e has a semicircular cross-section and is positioned at the end of the tongue piece 32d far away from its support, it can easily climb over the projection 32e even if the manipulation knob 34b is manipulated by applying light loads, and in addition, it can be easily returned to the position in which action of re-exposing the needle 10 is taken.

FIG. 5(E) shows the state in which the manipulation piece 34 has moved to the really stopping position. The really stopping position is formed in such a manner as to hold the manipulation piece 34 between the two projections 32f and make it hard to return them to the temporarily stopping position or the position in which action of re-exposing the needle 10 is taken. One of the real-stopping projections 32f, specifically the projection 32f on the temporarily stopping position side is shaped in such a manner as to have a curved surface on the temporarily stopping position side and a vertical surface on the really stopping position side so that the manipulation piece 34 can be easily moved from the side near to the temporarily stopping position to the really stopping position, but not from the really stopping position to the side near to the temporarily stopping position. Furthermore, since the real-stopping projections 32f are positioned near the supporting point of the tongue piece 32d, the tongue piece 32d is hard to deform.

In the embodiment shown in FIG. 5, the actual situation of the manipulation piece 34 relative to the needle 10 is easy to understand visually, in addition, manipulative easiness or hardness is realized by a simple structure.

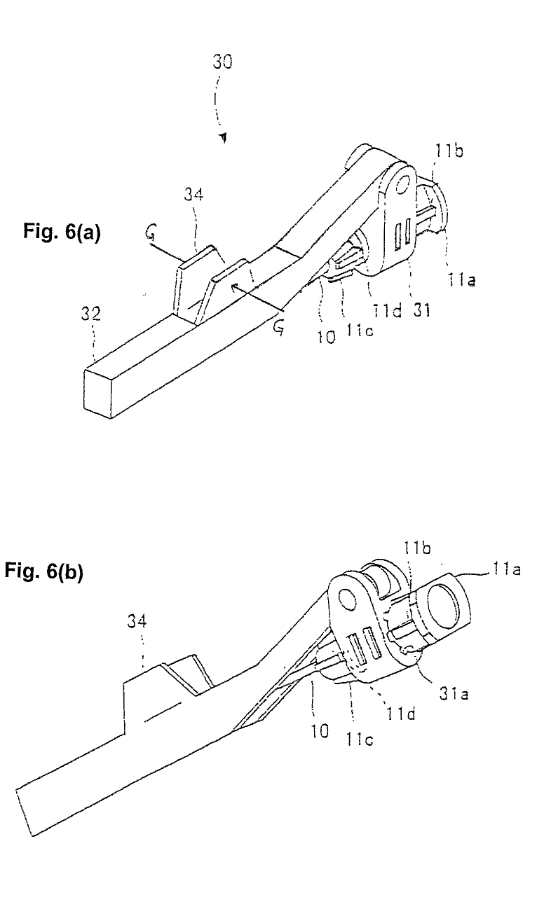

Then the present invention will be described with reference to FIG. 6. The needle covering member of this embodiment is formed in such a manner as to make it possible to select the state in which the needle hub 11 and the fixing portion 31 synchronize with the protector 32 when the protector 32 turns and the state in which the fixing portion 31 is idling, in addition, to make it possible to expose the needle 10 housed in the protector 32 again. In the following this embodiment will be described in further detail.

The needle hub includes a guard portion 11a formed at its end opposite to the needle 10; a cylindrical body slightly tapered from the guard portion 11a toward the needle 10; synchronizing projections 11b projecting from the body portion toward its periphery; slip-off preventers 11c projecting compared with the body portion toward its periphery; and an idling portion 11d formed between the synchronizing projection portion 11b and the slip-off preventer portion 11c.

Although the inside perimeter of the fixing portion 31 is larger than the outside perimeter of the idling portion lid on the hub, the fixing portion 31 having slipped through the idling portion 11d does not slip off due to the presence of the slip-off preventers 11c. When assembling the needle covering member, the needle hub and a syringe, the fixing portion 31 is subjected to elastic deformation and thereby allowed to climb over the slip-off preventer portion 11c from the needle 10 side.

First, the needle hub 11 of the needle 10 is fixed to the tip portion of a syringe, and then on the periphery of the needle hub 11 of the needle 10 the fixing portion 31 is fixed by friction engagement. When engaging the fixing portion 31 with the needle hub 11, the circular ring of the fixing portion 31 is subjected to slight deformation and thereby allowed to climb over the slip-off preventer portion 11c. Thus, an injector can be provided which is equipped with the needle covering member and hence of high safety. If the fixing portion 31 and the periphery of the needle hub 11 should be disengaged, the needle covering member 30 will not be separated from the needle hub 11 immediately.

When intending to turn to the tip surface of the needle in using the injector, if the fixing portion 31 is turned by engaging synchronizing hollows 31a with the synchronizing projections 11b, the needle hub 11 can turn in synchronization with the turning motion of the fixing portion; accordingly, the angle of the tip surface of the needle can be easily adjusted.

When intending to turn directly to the protector 32 alone not through the fixing portion 31, the fixing portion 31 is positioned at the idling portion 11d. As a result, the protector 32 does not come in contact with the synchronizing projection portion 11b, whereby the angle or position of the protector 32 alone can be adjusted.

The needle covering member includes a ring-shaped fixing portion 31 for fixing the needle covering member on the needle hub 11 of the needle 10, a protector 32 for housing and covering the needle 10, and a connecting portion (hinge) for connecting the protector 32 and the fixing portion 31 to each other.

The above protector 32 is a box-shaped protector with its underside opened, the opening formed in the underside is a housing opening for getting the needle 10 in and out of the protector 32 and the inside space of the housing opening 32a is a housing space. In the middle of the length of the surface opposite to the housing opening, provided is a manipulation opening which is in communication with the housing space. To the manipulation opening a pair of manipulation pieces 34 are provided. The pair of manipulation pieces 34 are hooky pieces with their hooks facing inside and their ends opposite to the hooks projecting from the housing space, and they are integrated into the protector 32 at the boundary between themselves and the manipulation opening.

The hooky parts described above function as a stopper portion capable of hooking the middle of the length of the needle 10 or its vicinity onto them when the protector 32 is turned relative to the needle 10 toward the housing opening side and covers the needle 10. The protector 32 is designed to allow the spacing between the two facing hooky parts to become smaller than the diameter of the needle 10 when the protector 32 is turned relative to the needle 10 toward the housing opening.

In the state where the stopper portion as described above hooks the middle of the length of the needle 10 or its vicinity, the protector 32 does not turn in such a direction as to expose the needle 10 even under external loads, unless the stopper portion or the needle 10 breaks. Since the needle-supporting portion 34a supports the needle 10 at a position away from the fixing portion 31 around which the protector 32 turns, the loads applied to the stopper portion can be reduced, particularly when external loads are applied to the tip portion of the protector 32 or its vicinity, compared with the structures in which the protector 32 is stopped near the fixing portion 31, which is in the vicinity of the root of the protector; as a result, the needle tip can be more effectively prevented from being exposed. The needle covering member of this embodiment contributes to reducing the consumption of raw materials because it adopts the above described structure.

Further, though not shown in the figure, the needle covering member of this embodiment includes a needle contacting portion 35 as a needle-stabilizing-functioning portion for stabilizing the needle 10 in such a position as to make it easier to release the protector 32 from the stopped state. Although the housing space is large for the thickness of the needle 10, the presence of the needle contacting portion 35 enables positioning of the needle 10 housed in the housing space 32b without offsetting.

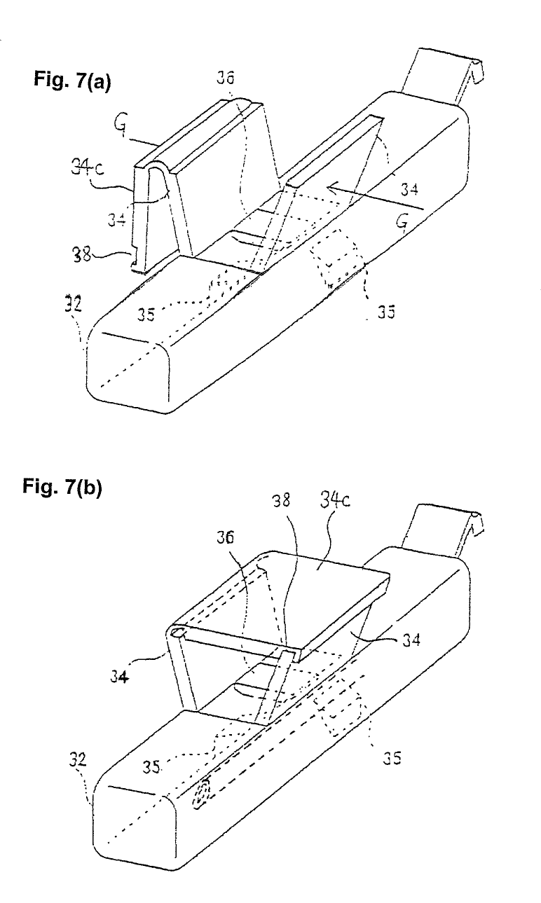

The embodiment described above can be provided as an assemblage of an unused needle 10 and a needle covering member, and the assemblage can be packed in such a state that the protector 32 of the needle covering member covers the needle 10 and the stopper portion prevents the needle 10 from being exposed. This makes it unnecessary to protect the needle with a separate member such as cap, leading to reduction of raw materials and wastes accompanying cap manufacture. In this embodiment, since the manipulation flow lines G, which indicate the direction in which loads are applied, are not on a plane H on which the protector turns, the accidental exposure of the needle point can be prevented effectively. In the embodiments shown in FIGS. 7 to 13 as well, since the manipulation flow lines G, which indicate the direction in which loads are applied, are not on a plane H on which the protector turns, the accidental exposure of the needle point can be prevented effectively. And also in these embodiments, the manipulation flow lines G are perpendicular to the plane H on which the protector turns.

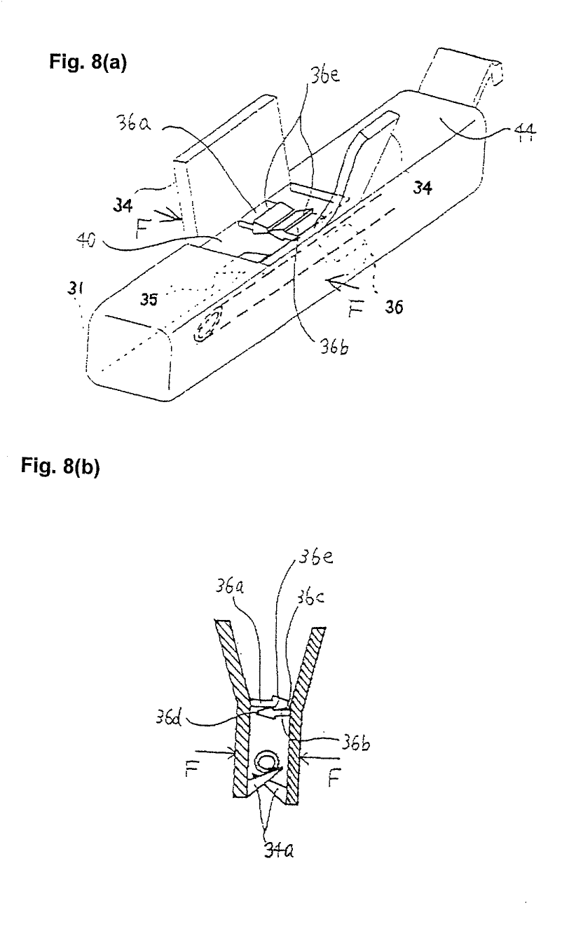

FIGS. 7 and 8 shows other variations of really stopping portion.

The needle covering member shown in FIGS. 7(A) and (B) includes a connecting plate 34c as a release-retarding-functioning portion, which is provided on one of the upper ends of the projecting parts of the pair of manipulation pieces 34 via a foldable hinge. The connecting plate 34c is provided with an engaging groove 38 into which the opposite manipulation piece 34 without a connecting plate is put when bringing the protector 32 to the really stopped state. This retards the two projecting parts approaching each other.

In the needle covering member shown in FIG. 8, atop surface opening 40 is provided in the top surface 44 of the protector, and in the above opening, auxiliary manipulation pieces 36a, 36b as a release-retarding-functioning portion are provided from the two facing side surfaces of the protector 32 in such a manner as to face inside. The tips of the auxiliary manipulation pieces 36a, 36b are provided with plane portions 36c, 36d, respectively, and between the plane portions 36c, 36d and the roots of the auxiliary manipulation pieces 36a, 36b provided are projection-like engaging portions 36e capable of stopping each other in engagement.

In order to release the protector 32 from the stopped state to which it has been brought by the needle-supporting portion 34a as a stopper portion, first the “right” and “left” manipulation pieces 34 are pushed from both their sides. This allows the auxiliary manipulation pieces 36a, 36b to move inward. Then the plane portions 36c, 36d provided on the auxiliary manipulation pieces 36a, 36b collide with each other. After the collision, the point at which the collision has occurred serves as a fulcrum, and with the point as a fulcrum the sides of the protector open sideways, allowing the needle-supporting portions 34a to open outward. Thus the needle is released from the protector.

When it becomes unnecessary to release the needle from the protector and the needle is finally discarded, both sides of the protector are directly pushed in the direction shown by F of FIG. 8 (B). When a certain degree of loads are applied thereto, the auxiliary manipulation pieces 36a, 36b climb over each other's engaging portions 36e. Once the auxiliary manipulation pieces 36a, 36b have climbed over the portions, the movement of the protector in the reverse direction is retarded and the movement of the needle-supporting portion 34a as well; thus, the needle 10 is held inside the protector 32 and prevented from being exposed again.

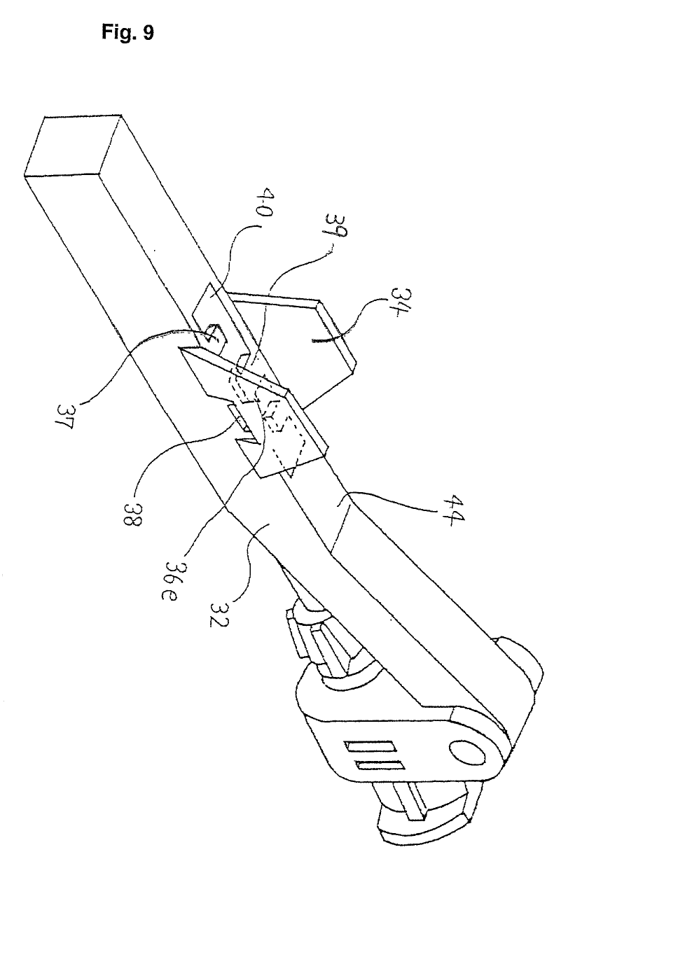

The needle covering member shown in FIG. 9 is the one as shown in FIG. 6 except that it further includes a release-retarding-functioning portion. The release-retarding-functioning portion includes an engaging piece 39 and an engaging groove 38. At the tip of the engaging piece 39 is provided a laterally projecting engaging portion 36e. In the top surface 44 of the protector 32 is provided a rectangular top surface opening 40, and the engaging piece 39 is provided on one longer side of the rectangular top surface opening 40 and the engaging groove 38 on the other longer side of the same in such a position as to face the engaging piece 39. On each longer side of the top surface opening 40 is provided a contact portion 37. And from both side surfaces of the protector 32, manipulation pieces 34 part of which is provided with a window extend upward at an angle.

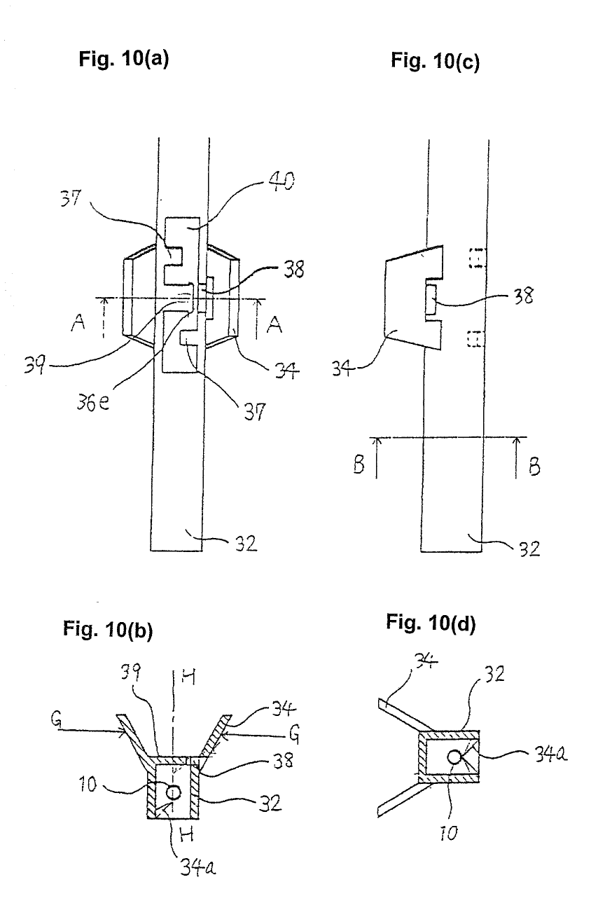

The state in which a needle is covered with a protector 32 is shown in FIGS. 10 (A) to (D). In such a state, the needle 10 is supported by the needle-supporting portion 34a and prevented from being exposed accidentally to the outside of the protector 32. FIG. 10 (B) is a view in section along the line A-A showing the needle covering member of FIG. 10(A), FIG. 10(C) is a side view showing the needle covering member of FIG. 10(A), and FIG. 10(D) is a view in section along the line B-B showing the needle covering member of FIG. 10 (A).

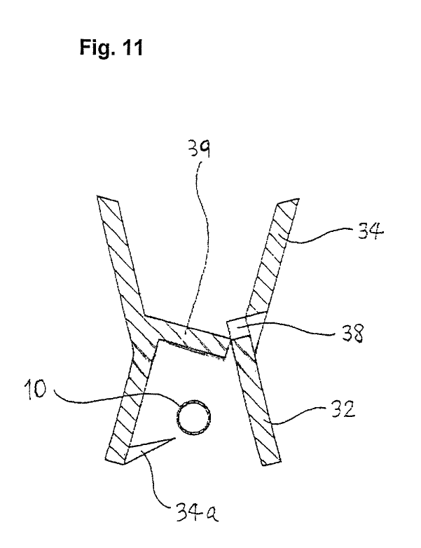

In order to release the needle 10 from the state in which it is supported by the needle-supporting portion 34a and expose it again, both sides of the protector 32 are opened sideways by holding the manipulation pieces 34 and pushing it from both sides inward, just like other embodiments. In this case, the engaging portion 36e of the engaging piece 39 is not put into the engaging groove 38 and therefore the manipulation pieces are not stopped in engagement. The engaging piece 39 turns when the manipulation pieces 34 are pushed inward as shown in FIG. 11. Accordingly, the position of the engaging piece 39 does not coincide with that of the engaging groove 38. Thus the engaging piece 39 is not stopped in engagement with the engaging groove 38 and collides against the inside wall of one side of the protector 32. Once the engaging piece 39 collides against the inside wall of the protector 32, the point of the collision serves as a fulcrum when opening the needle-supporting portion 34a.

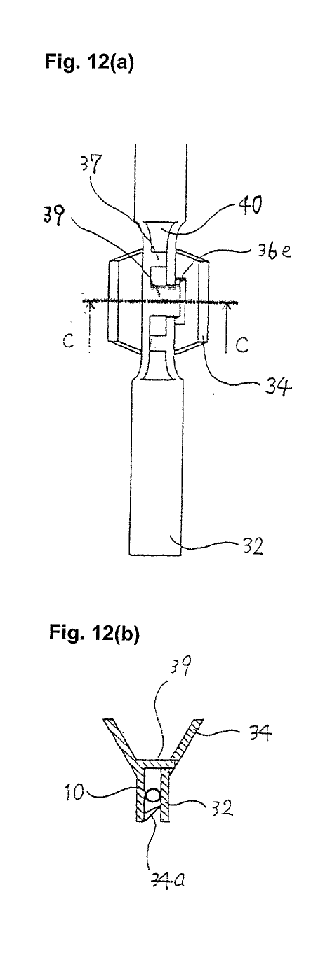

When it becomes unnecessary to release the needle from the protector and the needle is finally discarded, not the manipulation pieces 34, but the protector 32 is directly pushed from both its sides as shown in FIG. 12. FIG. 12 (B) is a view in section along the line C-C showing the needle covering member of FIG. 12(A). The protector 32 is easily crushed when pushed, partly because its strength is partially decreased due to the presence of the top surface opening 40. At the time of crushing, the engaging piece 39 is put into the engaging groove 38. Once the engaging piece 39 has been put into the engaging groove 38, the movement of the engaging piece 39 in the returning direction is retarded due to the engaging portion 36e provided at the tip of the engaging piece 39; thus, the needle is kept held in the protector 32. The engaging groove 38 is not necessarily a groove, but it maybe a hole. The engaging piece 39 shown in the figures is in the form of a plate, but it may also be in the form of a cylinder, a prism or the like.

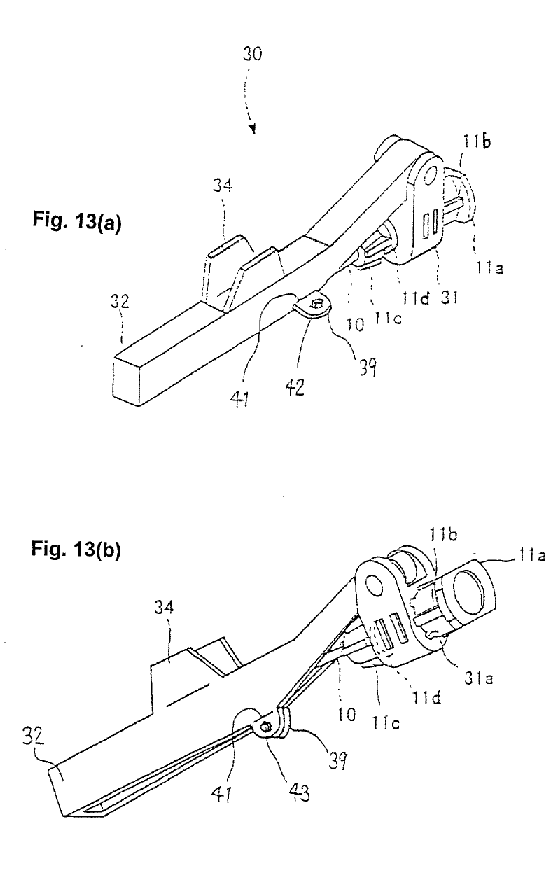

The needle covering member shown in FIG. 13 is the same as those of the other embodiments except its release-retarding-functioning portion; accordingly, the release-retarding-functioning portion alone will be described. The release-retarding-functioning portion consists of a pair of engaging pieces 39. The engaging pieces 39 are formed at the lower ends of both sides of the protector 32 via a hinge 41 in such a manner as to be foldable. In one of the engaging pieces 39 is provided an engaging hole 42 and on the other engaging piece 39 an engaging projection 43. Around the engaging hole 42 a groove is provided so as for the engaging projection to easily go into the hole 42. The engaging projection 43 is mushroom-shaped, though not shown in the figure, and its tip portion is tapered in such a manner that the diameter becomes smaller toward the tip; thus, the engaging projection 43 is easy to get in the engaging hole 42, but difficult to get out of it.

### INDUSTRIAL APPLICABILITY

According to the present invention, a needle covering member can be provided, which can protect the needle tip when carried to a patient with its syringe containing fluid to be injected and which can retard re-exposure of the needle covered with a protector even under external loads, and a syringe equipped with the above needle covering member can also be provided.

What is claimed is:

1. A needle covering member comprising:
a fixing portion for fixing the needle covering member in the vicinity of the tip of a syringe;
a protector for housing and covering a needle; and
a connecting portion for connecting the protector (32 and the fixing portion to each other;
wherein the protector comprises:
a housing opening through which the needle gets in and out;
a stopper portion for preventing the protector from turning in such a direction as to expose a needle even under external loads when the protector is turned toward the housing opening and covers the needle; and
a manipulation piece for releasing the protector from the stopper portion so that the needle covered with the protector may be exposed through the housing opening to be used; and
wherein the stopper portion is formed at an end of the manipulation piece near to the housing opening and intersects the needle or a needle hub in the interior of the protector.

2. The needle covering member according to claim 1, wherein the protector deforms elastically so that the needle may easily get out of the housing opening when manipulating the manipulation piece to release the stopped state by the stopper portion.

3. The needle covering member according to claim 1, including a needle-stabilizing-functioning portion for housing the needle in the interior of the protector in a position where the needle is easily released.

4. The needle covering member according to claim 1, wherein the protector includes a release-retarding-functioning portion for retarding the release of the stopped state by the manipulation piece.

5. The needle covering member according to claim 1, wherein the stopper portion intersects the needle around the middle of its length in the interior of the protector.

6. The needle covering member according to claim 1, wherein the manipulation flow line, which indicates the direction in which loads are applied to the manipulation piece so as to release the stopped state by the stopper portion, is not on a plane on which the protector turns.

7. A syringe equipped with the needle covering member according to claim 1, including:
a piston which enables the discharge and suction of fluid;
a barrel portion for holding the fluid; and
a tip portion provided at the tip of the barrel portion.

8. A syringe equipped with the needle covering member according to claim 2, including:
a piston which enables the discharge and suction of fluid;
a barrel portion for holding the fluid; and
a tip portion provided at the tip of the barrel portion.

9. A syringe equipped with the needle covering member according to claim 3, including:
a piston which enables the discharge and suction of fluid;
a barrel portion for holding the fluid; and
a tip portion provided at the tip of the barrel portion.

10. A syringe equipped with the needle covering member according to claim 4, including:
a piston which enables the discharge and suction of fluid;
a barrel portion for holding the fluid; and
a tip portion provided at the tip of the barrel portion.

11. A syringe equipped with the needle covering member according to claim 5, including:
a piston which enables the discharge and suction of fluid;
a barrel portion for holding the fluid; and
a tip portion provided at the tip of the barrel portion.

12. A syringe equipped with the needle covering member according to claim 6, including:
a piston which enables the discharge and suction of fluid;
a barrel portion for holding the fluid; and
a tip portion provided at the tip of the barrel portion.
